(19) United States
(12) Patent Application Publication
(10) Pub. No.: US 2012/0288848 A1
(43) Pub. Date: Nov. 15, 2012

(54) Multi-Dimensional Flow Pad Technology for Covering Three-Dimensional Dome Shaped Anatomies

(75) Inventors: Jeffrey W. Latham, Buda, TX (US); Randall D. Manteufel, San Antonio, TX (US); Nolan P. Manteufel, San Antonio, TX (US)

(73) Assignee: TRAUMATEC, INC., Houston, TX (US)

(21) Appl. No.: 13/354,824

(22) Filed: Jan. 20, 2012

Related U.S. Application Data
(60) Provisional application No. 61/434,784, filed on Jan. 20, 2011.

(51) Int. Cl. A61F 7/12; A01N 1/02; A61F 7/10; A61F 7/08; A61M 5/44

(52) U.S. Cl. 435/1.1; 607/104; 607/105; 604/113

(57) ABSTRACT

A three dimensional dome wrap or pad for direct patient surface tissue applications for symmetrical dome shaped anatomy and extremities is disclosed herein. The pad of the present invention comprises a system of smooth streamline mathematically configured channels throughout one or more similarly shaped panels or lobes bonded distinctively to make one unit for the purpose of temperature management of three dimensional objects or subjects when it is applied or inserted.

### CROSS-REFERENCE TO RELATED APPLICATIONS

This application claims the benefit of U.S. provisional patent application No. 61/434,784, filed Jan. 20, 2011 the contents of which are incorporated by reference herein.

### TECHNICAL FIELD OF THE INVENTION

The present invention relates in general to padding and protective articles, and more particularly, to a three dimensional dome wrap or pad for direct patient surface tissue applications for symmetrical dome shaped anatomy and extremities.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with design and uses of articles for cushioning, protecting, and providing temperature control to various body protrusions, organs, and muscle areas.

U.S. Pat. No. 5,840,397 issued to Landi and Wilson (1998) describes an improved sports pad for protecting a particular area of a user's body and including a relatively rigid, lightweight plate molded to conform to the protected body area, an outer layer of resilient thermoplastic honeycomb padding affixed to the outer surface of the plate, and an inner layer of resilient thermoplastic honeycomb padding affixed to the inner surface of the plate. The outer layer is preferably of a closed-cell configuration while the inner layer is of an open-celled configuration perforated to allow the flow of air from cell to cell.

U.S. Pat. No. 5,891,187 issued to Winthrop and Bayron (1999) discloses a heating or cooling pad device, particularly for use in surgical operations and medical procedures, which has a human shaped pad for receiving a lying body. The heating or cooling pad can be formed from a chemical reaction pack to generate heat or cold. Alternatively, the pad may consist of an inflatable structure which conducts thermally controlled pressurized fluid or gas from an inlet port through a series of interacting channels. A plurality of holes across the patient-facing surfaces of the pad and patient-wrapping flaps facilitate escape of pressurized gases. For fluids, a corresponding outlet port is provided. For electrical elements, a power source is connected. The pad might also have a concave curvature on its surface to thereby cradle a patient when inflated. A stiffener might be adhered to the lower surface of the pad. The pad includes a series of hook and pile attachment strips for selectively attaching modular sections of insulated material which can then be used to wrap around and secure the patient lying on the pad. A single piece section of insulated material might also be used underneath the pad, with the section having flaps which extend around to secure and insulate the patient. Additionally, separate torso and headpiece sections of insulated material can be used to facilitate selective access to various parts of the patient, and yet optimize thermal energy conversation in and around the patient.

U.S. Pat. No. 5,894,615 issued to Alexander (1999) discloses to a bed pad with an embedded circuit of continuous tubing. Portable heating and refrigerating means are operatively connected to a second tubing circuit by quick disconnect couplings. The Alexander patent uses electrical control methods to selectively operated to heat or cool the liquid in said second tubing circuit. Thermostatic controls may optionally be applied to both the heating and refrigeration. This has particular use in surgical operating rooms for raising and lowering the temperature of the patient. It is also useful in the recovery room, hospital and or convalescent home.

U.S. Patent Application No. 20090287060 (Pell and Crenshaw, 2009) discloses methods and devices are disclosed to reduce the tissue trauma that occurs when a physician retracts or otherwise deforms a patient's tissues for surgery or other medical procedures. In one part, methods and devices are disclosed for cooling the tissue around the incision. In another part, methods and devices are disclosed that elute drugs into the tissues of the tissue margin. In another part, methods and devices are disclosed to engage tissues during retraction to cushion, to sense tissue state, and to modulate tissue state.

### SUMMARY OF THE INVENTION

The present invention describes a flow pad for covering three-dimensional dome shaped anatomies or objects for the purposes of temperature management when applied to or inserted in the object. The pad of the present invention comprises a system of smooth streamline mathematically configured channels containing a heating/cooling fluid that is circulated for the purposes of temperature control.

A multi-dimensional pad for covering an object, an organ, a tissue, an anatomical part, for insertion into a body organ or tissue or both for providing a temperature control, a temperature therapy, prevent trauma or any combinations thereof is described in one embodiment of the present invention. The pad comprises: (i) one or more axisymmetric or similarly shaped surfaces, lobes or panels that are permanently or removeably bonded together to form the pad; wherein the surfaces, lobes or the panels comprise an interior and an exterior, wherein the interior is in direct contact with the object, the organ, the tissue or the anatomical part, (ii) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (iii) one or more hose connectors attached to the interior in communication with the network, wherein the hose connectors are connected to and deliver the heating or the cooling fluid from a pump or a fluid reservoir to the network, and (iv) one or more fasteners, wherein the fasteners are capable of fastening on to other fasteners or regions on the interior or exterior of the pad to cover the object, the organ, the tissue or the anatomical part.

The multi-dimensional pad described hereinabove further comprises: one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof, an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part, and one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part.

In one aspect of the pad of the present invention the air pad is capable of being inflated to provide pressure to the multi-dimensional pad. In another aspect the air pad comprises one or more valves for inflation, deflation or both to provide intermittent or varying pressures to the multi-dimensional pad. In yet another aspect the pad comprises split networks or channels to provide temperature control or localized heating or cooling to specific areas of the organ, the tissue or the anatomical part. The network described herein is further defined as comprising two networks, wherein the heating fluid and the cooling fluid independently circulate in the two networks concurrently to provide simultaneous heating and cooling to a same or a different area of the organ, the tissue or the anatomical part.

In one aspect of the pad described hereinabove the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof. In another aspect the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes. The multi-dimensional pad of the present invention can be used to cover an anatomical object selected from the group consisting of an arm, a shoulder, a knee, a head, a neck, a back, a hip, ankles, an elbows and hands. In yet another aspect the multi-dimensional pad provides temperature control to one or more objects selected from the group consisting of intravenous (IV) bags, medications, surgical equipment, hoses, tubes, and any combinations thereof by covering the object to form a thermal sleeve. In another aspect the multi-dimensional pad can be inserted into a bone, the organ, a brain surface, and a spinal cord canal for cooling cerebral spinal fluid or for providing temperature control.

In another embodiment the instant invention discloses a multi-dimensional pad for covering an object, an organ, a tissue, an anatomical part, for insertion into a body organ or tissue or both for providing a temperature control, a temperature therapy, prevent trauma or any combinations thereof comprising: (i) one or more axisymmetric or similarly shaped surfaces, lobes or panels that are permanently or removeably bonded together to form the pad; wherein the surfaces, lobes or the panels comprise an interior and an exterior, wherein the interior is in direct contact with the object, the organ, the tissue or the anatomical part, (ii) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (iii) one or more secondary flow paths, crossovers or bypasses for uninterrupted fluid flow in case of a blockage, pressure build up or obstructions in the primary channels, conduits or tubes, (iv) one or more hose connectors attached to the interior in communication with the network, wherein the hose connectors are connected to and deliver the heating or the cooling fluid from a pump or a fluid reservoir to the network, (v) one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof, (vi) an air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part, (vii) one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part, and (viii) one or more fasteners, wherein the fasteners are capable of fastening on to other fasteners or regions on the interior or exterior of the pad to cover the object, the organ, the tissue or the anatomical part.

Yet another embodiment of the present invention provides a method for providing a controlled temperature environment to an organ, a tissue or an anatomical part of a subject comprising the steps of: identifying the subject in need of the controlled temperature environment; providing a multi-dimensional pad comprising: (a) one or more axisymmetric or similarly shaped surfaces, lobes or panels that are permanently or removeably bonded together to form the pad, wherein the surfaces, lobes or the panels comprise an interior and an exterior, wherein the interior is in direct contact with the object, the organ, the tissue or the anatomical part, (b) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (c) one or more hose connectors attached to the interior in communication with the network, wherein the hose connectors are connected to and deliver the heating or the cooling fluid from a pump or a fluid reservoir to the network, and (d) one or more fasteners, wherein the fasteners are capable of fastening on to other fasteners or regions on the interior or exterior of the pad to cover the object, the organ, the tissue or the anatomical part; placing the organ, the tissue or the anatomical part in the multidimensional pad or covering, wrapping or overlaying the multidimensional pad over the organ, the tissue or the anatomical part such that the interior is in direct contact with the organ, the tissue or the anatomical part; fastening the one or more fasteners on the interior of the pad to one or more fasteners or regions on the interior or exterior of the pad to obtain a snug fit of the multidimensional pad over the organ, the tissue or the anatomical part; and providing a controlled temperature by operating the pump or the fluid reservoir to deliver the circulating heating or cooling fluid to the network through the one or more hose connectors and associated tubing.

The pad used in the method as described hereinabove further comprises: one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof; an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the organ, the tissue or the anatomical part, and one or more additional layers attached removeably or permanently to the multi-dimensional pad comprising air, fluid, medication or any combinations thereof, wherein the additional layers are separate from the flow channels and are in direct contact with the organ, the tissue or the anatomical part.

In one aspect of the present invention the air pad is capable of being inflated to provide pressure to the multi-dimensional pad. In another aspect the air pad comprises one or more valves for inflation, deflation or both to provide intermittent or varying pressures to the multi-dimensional pad. In yet another aspect the multi-dimensional pad curls up to provide a more snug fit over the object, the organ, the tissue or the anatomical part when fluid circulation is initiated. The pad used in the method of the present invention comprises split networks or channels to provide temperature control or localized heating or cooling to specific areas of the organ, the tissue or the anatomical part, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes. The network in the pad of the present invention is further defined as comprising two networks, wherein the heating fluid and the cooling fluid independently circulate in the two networks concurrently to provide simultaneous heating and cooling to a same or a different area of the organ, the tissue or the anatomical part. In one aspect the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof. In another aspect the anatomical object is selected from the group consisting of an arm, a shoulder, a knee, a head, a neck, a back, a hip, ankles, an elbows and hands. In yet another aspect the multi-dimensional pad can be inserted into a bone, the organ, a brain surface, and a spinal cord canal for cooling cerebral spinal fluid or for providing temperature control.

In yet another embodiment the instant invention discloses a method for providing a controlled temperature environment to an object comprising the steps of: (i) providing a thermal sleeve comprising: (a) one or more axisymmetric or similarly shaped surfaces, lobes or panels that are permanently or removeably bonded together to form the sleeve, wherein the surfaces, lobes or the panels comprise an interior and an exterior, wherein the interior is in direct contact with the object, the organ, the tissue or the anatomical part, (b) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (c) one or more hose connectors attached to the interior in communication with the network, wherein the hose connectors are connected to and deliver the heating or the cooling fluid from a pump or a fluid reservoir to the network, and (d) one or more fasteners, wherein the fasteners are selected from the group consisting of Velcro fasteners, hooks and loops, button, pin, and any combinations thereof; (ii) placing the object in the thermal sleeve or covering, wrapping or overlaying the thermal sleeve over the such that the interior is in direct contact with the object; (iii) fastening the one or more fasteners on the interior of the sleeve to one or more fasteners or regions on the interior or exterior of the pad to obtain a snug fit of the sleeve over the object; and (iv) providing a controlled temperature by operating the pump or the fluid reservoir to deliver the circulating heating or cooling fluid to the network through the one or more hose connectors and associated tubing.

The thermal sleeve described in the method hereinabove further comprises: one or more open-face holes, curves, lobe like designs or any combinations thereof on the surface, lobes or the panels, wherein the open-face holes, curves, lobe like designs assist in a fitting of the sleeve on oddly shaped objects, projections, protrusions or any combinations thereof, an optional air-flow mechanism comprising one or more secondary flow channels or an air pad attached removeably or permanently to the thermal sleeve, wherein the secondary flow channel or the air-pad provides a sustained, intermittent or varying pressures to the thermal sleeve to enhance contact of the multi-dimensional pad with the object, and one or more additional layers attached removeably or permanently to the thermal sleeve comprising air, fluid, medication or any combinations thereof, wherein the additional layers are separate from the flow channels and are in direct contact with the object. In one aspect the air pad is capable of being inflated to provide pressure to the thermal sleeve. In another aspect the air pad comprises one or more valves for inflation, deflation or both to provide intermittent or varying pressures to the thermal sleeve. In yet another aspect the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes. In another aspect the object is selected from the group consisting of intravenous (IV) bags, medications, surgical equipment, hoses, tubes, and any combinations thereof.

A method of preventing or minimizing brain trauma following a head injury, a brain surgery, a stroke, an aneurysm, a hemorrhage or any combinations thereof in a subject is described in one embodiment of the present invention. The method comprises the steps of:

identifying the subject in need of prevention or minimization of the brain trauma; placing the head of the subject or placing a multi-dimensional pad under the head of the subject, wherein the position of the pad is such that the head of the subject is at a center of the pad, wherein the pad comprises: (a) one or more axisymmetric or similarly shaped surfaces, lobes or panels that are permanently or removeably bonded together to form the pad, wherein the surfaces, lobes or the panels comprise an interior and an exterior, wherein the interior is in direct contact with the head, (b) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (c) one or more hose connectors attached to the interior in communication with the network, wherein the hose connectors are connected to and deliver the heating or the cooling fluid from a pump or a fluid reservoir to the network, and (d) one or more fasteners, wherein the fasteners are capable of fastening on to other fasteners or regions on the interior or exterior of the pad to cover the head; fastening the one or more fasteners on the interior of the pad to one or more fasteners or regions on the interior or exterior of the pad to obtain a snugly fit cap of the multidimensional pad covering the head and neck of the subject; and operating the pump or the fluid reservoir to deliver the circulating heating or cooling fluid to the network through the one or more hose connectors and associated tubing to provide a controlled temperature environment to minimize or prevent the brain trauma.

The multi-dimensional pad of the present invention further comprises: one or more open-face holes, curves, lobe like designs or any combinations thereof on the surface, lobes or the panels, wherein the open-face holes, curves, lobe like designs assist in a fitting of the pad to the head, an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the head, and one or more additional layers attached removeably or permanently to the multi-dimensional pad comprising air, fluid, medication or any combinations thereof, wherein the additional layers are separate from the flow channels and are in direct contact with the head.

In one aspect of the method of the present invention the air pad is capable of being inflated to provide pressure to the thermal sleeve. In another aspect the air pad comprises one or more valves for inflation, deflation or both to provide intermittent or varying pressures to the thermal sleeve. In yet another aspect the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

In yet another embodiment the instant invention relates to a method of minimizing or preventing degradation of an organ during harvesting, transporting, transplanting into a patient or any combinations thereof comprising: (i) placing the organ on a multi-dimensional pad or thermal sleeve, wherein the pad or the sleeve comprises: one or more axisymmetric or similarly shaped surfaces, lobes or panels that are permanently or removeably bonded together to form the pad or the sleeve, wherein the surfaces, lobes or the panels comprise an interior and an exterior, wherein the interior is in direct contact with the object, the organ, the tissue or the anatomical part, one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, one or more hose connectors attached to the interior in communication with the network, wherein the hose connectors are connected to and deliver the heating or the cooling fluid from a pump or a fluid reservoir to the network, and one or more fasteners, wherein the fasteners are capable of fastening on to other fasteners or regions on the interior or exterior of the pad or the sleeve to cover the organ, (ii) fastening the one or more fasteners to obtain a snug fit around the organ to cover or overlay the organ, and (iii) operating the pump or the fluid reservoir to deliver the circulating cooling fluid to the network through the one or more hose connectors to provide a controlled temperature environment to minimize or prevent degradation during organ harvest, transport, transplant or any combinations thereof.

The thermal pad of the method above further comprises one or more open-face holes, curves, lobe like designs or any combinations thereof on the surface, lobes or the panels, wherein the open-face holes, curves, lobe like designs assist in a fitting of the pad or the sleeve on oddly shaped organs, projections, protrusions or any combinations thereof, an optional air-flow mechanism comprising one or more secondary flow channels or an air pad attached removeably or permanently to the thermal sleeve, wherein the secondary flow channel or the air-pad provides a sustained, intermittent or varying pressures to the thermal sleeve to enhance contact of the pad or the sleeve with the organ, and one or more additional layers attached removeably or permanently to the pad or the thermal sleeve comprising air, fluid, medication or any combinations thereof, wherein the additional layers are separate from the flow channels and are in direct contact with the organ.

A system for providing a controlled temperature environment during a surgical procedure, in an operation theater, in an emergency room or any combinations thereof is described in one embodiment of the present invention. The system comprises one or more thermal sleeves and multi-dimensional pads, wherein the thermal sleeves and multi-dimensional pads comprise: (a) one or more axisymmetric or similarly shaped surfaces, lobes or panels that are permanently or removeably bonded together to form the pad, wherein the surfaces, lobes or the panels comprise an interior and an exterior, wherein the interior is in direct contact with an object, an organ, a tissue or an anatomical part; (b) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid; (c) one or more hose connectors attached to the interior in communication with the network, wherein the hose connectors are connected to and deliver the heating or the cooling fluid from a pump or a fluid reservoir to the network; and (d) one or more fasteners, wherein the fasteners are capable of fastening on to other fasteners or regions on the interior or exterior of the pad to cover or overlay the object, the organ, the tissue or the anatomical part.

The system of the present invention further comprises: one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof, an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part, and one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part. In one aspect the air pad is capable of being inflated to provide pressure to the multi-dimensional pad. In another aspect the air pad comprises one or more valves for inflation, deflation or both to provide intermittent or varying pressures to the multi-dimensional pad. In yet another aspect the pad comprises split networks or channels to provide temperature control or localized heating or cooling to specific areas of the organ, the tissue or the anatomical part, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

In one aspect the multi-dimensional pad covers or overlays one or more anatomical objects selected from the group consisting of an arm, a shoulder, a knee, a head, a neck, a back, a hip, ankles, an elbows and hands during a surgical procedure. In another aspect of the system the thermal sleeve covers or overlays one or more objects selected from the group consisting of intravenous (IV) bags, medications, surgical equipment, hoses, tubes, and any combinations thereof. In yet another aspect the multi-dimensional pad can be inserted into a bone, the organ, a brain surface, and a spinal cord canal for cooling cerebral spinal fluid or for providing temperature control during a surgical procedure.

A method of providing a controlled temperature for one or more surgical and associated equipments, organs, tissues, and any combinations thereof during a surgical procedure comprising the step of wrapping, covering or overlaying with a multi-dimensional pad or a thermal sleeve is provided in one embodiment of the present invention.

The multi-dimensional pad or thermal sleeve described above comprises: (i) one or more axisymmetric or similarly shaped surfaces, lobes or panels that are permanently or removeably bonded together to form the pad, wherein the surfaces, lobes or the panels comprise an interior and an exterior, wherein the interior is in direct contact with the one or more surgical and associated equipments organs, tissues, and any combinations thereof, (ii) one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid, (iii) one or more hose connectors attached to the interior in communication with the network, wherein the hose connectors are connected to and deliver the heating or the cooling fluid from a pump or a fluid reservoir to the network, and (iv) one or more fasteners, wherein the fasteners are capable of fastening on to other fasteners or regions on the interior or exterior of the pad to cover the one or more surgical and associated equipments, organs, tissues, and any combinations thereof

In one aspect of the method the one or more surgical and associated equipments are selected from the group consisting of intravenous (IV) bags, medications, surgical equipment, hoses, tubes, and any combinations thereof. In another aspect of the method the multi-dimensional pad covers or overlays one or more anatomical objects selected from the group consisting of an arm, a shoulder, a knee, a head, a neck, a back, a hip, ankles, an elbows and hands or can be inserted into a bone, the organ, a brain surface, and a spinal cord canal for cooling cerebral spinal fluid or for providing temperature control during a surgical procedure. In yet another aspect the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows the multi-dimensional flow pad system of the present invention;

FIG. 2 is a schematic showing the channel width specifications of the flow-pad system of the present invention;

FIG. 3 is a schematic showing the optional moveably attachable secondary flow channel to add airflow over the fluid flow pad;

FIG. 4 is a schematic representation of a split channel design to control temperature specific areas and joints of the user;

FIG. 5 is a schematic representation showing sharp turns (bypass) in a symmetrical pattern to have a secondary flow path to each channel in case of flow blockage and pressure build up in one area or channel;

FIGS. 6 and 7 schematically show different secondary flow paths to move fluid to the next channel throughout the pad in case of a blockage or obstructions;

FIG. 8 shows the open face holes in the flow-pad system of the present invention for a more perfect fitting over pointed areas of the body;

FIG. 9 shows the pad of the present invention covering or wrapped around a three-dimensional body area;

FIG. 10 shows the pad or the wrap of the present invention showing the upper section, the lower side and one of the sides;

FIG. 11 is a schematic showing the use of the pad of the present invention to thermally control intravenous (IV) bags or transplant organs by forming a thermal sleeve;

FIGS. 12A-12H show the step by step instructions on the use of the thermal pad system of the present invention to cover the head of the subject;

FIG. 13 is a photograph showing the use of the thermal pad system of the present invention to wrap a shoulder area of the subject;

FIG. 14 is a photograph showing the use of the thermal pad system of the present invention to wrap a knee joint (knee brace) of the subject; and

FIG. 15 is a photograph showing the use of the thermal pad system of the present invention to wrap around the thigh of the subject.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

The present invention describes a universally shaped pad to lay flat on a surface or cover a three-dimensional body area for the purposes of temperature control. The pad of the instant invention comprises a systematic network of one or more channels that adjust in height depending on fluid volume in the channel. The pad has a distinctive shape of lobes and channels for supreme flow stream speed and thermodynamics. The pad of the present invention curls when the flow is initiated. The pad flow design of the present invention may be used to thermally control intravenous (IV) bags, medications, hoses or tubes and designed in the form of a thermal sleeve system. The technology may be used to thermally wrap narrowly or oddly shaped prominences or sharply rounded objects to control temperature.

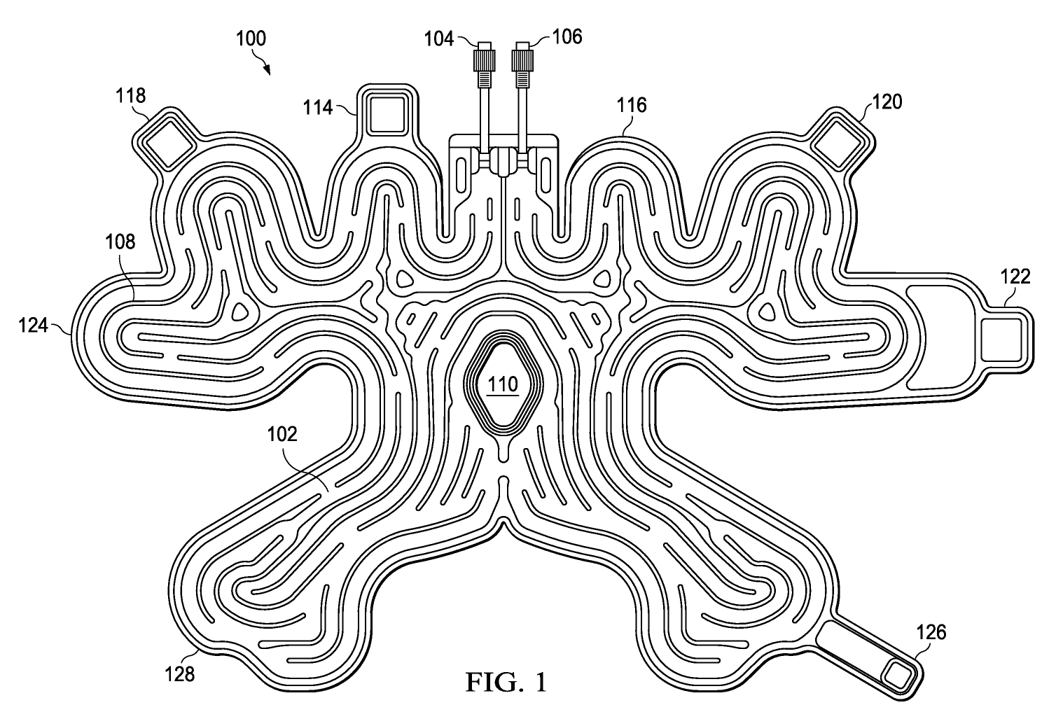

A guided multi-channel equilibrium flow path pad system designed to reduce flow drag and increase flow volume and speed while maximizing consistent thermodynamics throughout a system of smooth streamline mathematically configured channels throughout one or more similarly shaped panels or lobes bonded distinctively to make one unit for the purpose of temperature management of three dimensional objects or subjects when applied or inserted. The spacing of the flow channels also provides a way of maintaining full surface contact of curved objects as shown in FIG. 1.

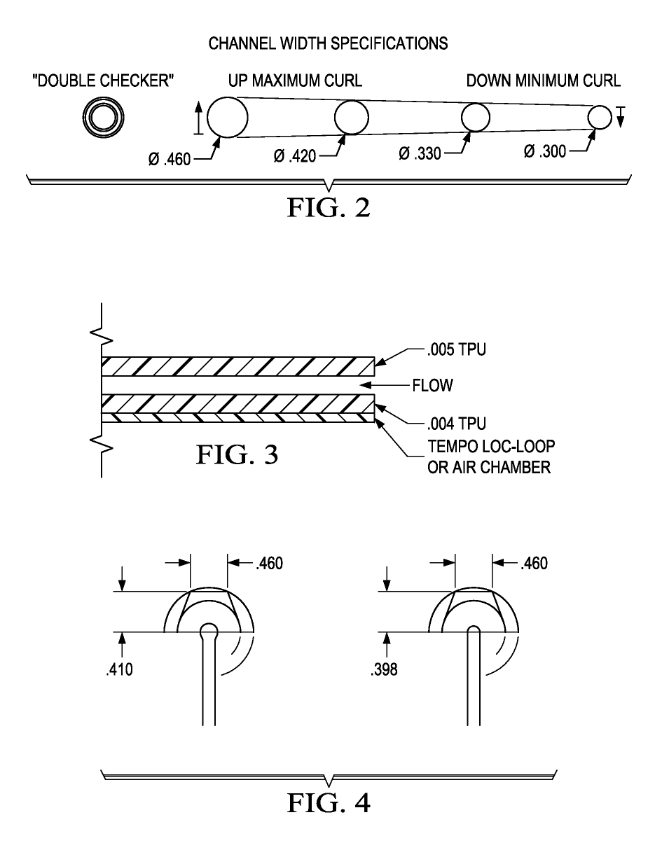

For example, the wider a channel is open, the more thermal fluid will adjust the height of the channel making it more capable of maintaining improved surface thermodynamics of a curved or circular body part. The narrowing of a channel increases the speed, pressure and decreases the volume of thermal fluids ability to flow in its channel and hereby adjusts the height of the channel to a lower setting thereby creating a three dimensional differential height of the pad for increased contact of curved objects once in place. The pad technology is designed to maintain full surface contact of a circular body part requiring temperature therapy for best fit (e.g. the fingers of the hand are each different sizes and thickness and so are the channels of the pad design) as seen in FIG. 2. The pad will curl when flow is initiated.

A secondary flow channel may be moveably attachable to the fluid pad to add airflow over an area that may provide gentle pressure over the fluid flow pad. The air pad may combine by billet welding of the fluid flow channels in order to maintain full contact with the surface of the pad and user (FIG. 3). The air pad may also permanently attach within the fluid flow pad intermittently delivering air pressure to conform both pads into a circular shape upon inflation. The air pad may also have the same shape as the fluid flow pad technology and have release valves within the pad to release pressure at an acceptable inflation rate. This may include a manual air pumping mechanism that holds consistent pressure, for e.g., blowing into the valve and close it like a flotation device.

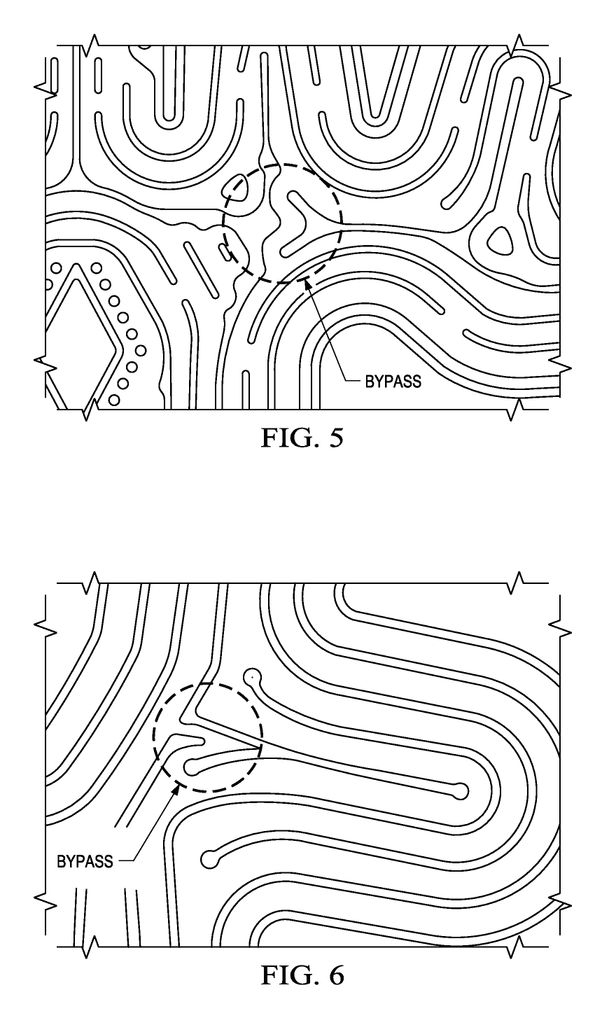

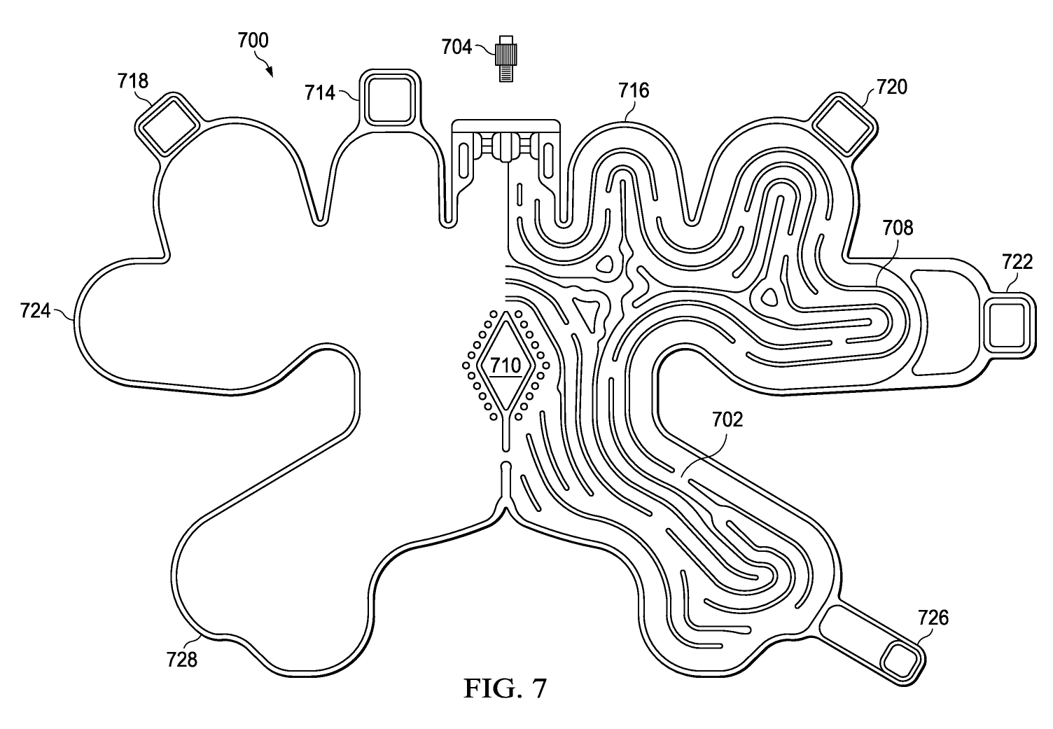

In one embodiment the flow path technology design of the wrap is split channeled to guide thermal fluid to provide an even pressurized push or pull within the pad to control temperature of specific areas and joints of the user (FIG. 4). The pad flow channels are designed to make sharp turns in a symmetrical pattern and have a secondary flow path to each channel in case of flow blockage and pressure build up in one area or channel (FIG. 5). A crossover channel is a secondary passageway to change flow into the adjacent channel. The flow patterns will automatically utilize the crossover channel gate by pressure blockage and the flow and build up pressure and move to the adjacent channel of least resistance maintaining the thermodynamics of the entire pad. The thermal fluid will continue to move water to the next channel consecutively from channel to channel throughout the pad upon any blockage. (FIGS. 5, 6, and 7).

The flow Channel pad technology can be used for a variety of applications such as direct patient surface tissue applications for symmetrical dome shaped anatomy and extremities. The technology may be used internally on organ tissue applications, organ transport, arterial insertion, infusion equipment and applications for temperature measurement and management of internal blood circulation, as seen in FIG. 7.

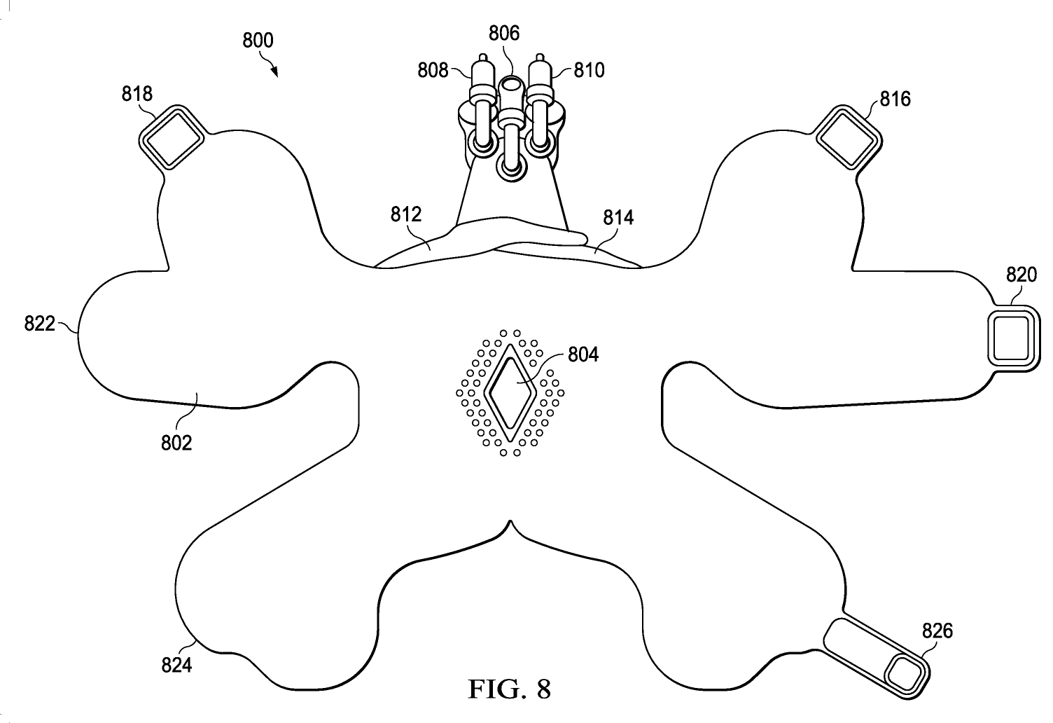

The three dimensional pad and or dome wrap is designed with open face holes for more perfect fitting over pointed areas of the body. The thermal pad may require being used on certain body parts that require good fitting and visualization of the areas being covered by the pad. This may allow for surgeons, nurses or healthcare providers to monitor skin tissues or for surgical purposes of having access to the patient. (FIG. 8)

A universally shaped pad to lay flat on a surface or cover a three-dimensional body area specifically tapered for covering most surface areas with a dome or joint for ease of application. The pad has a distinctive shape of lobes and channels for supreme flow stream speed and thermodynamics. The wrap has a lower section and an upper section with a left side and a right side when in place upon the subject or object. The wrap may have open face holes and curves in a lobe like design in order to overlay a dome or round shaped anatomy, for e.g., an arm, a shoulder, a knee, a head, a neck, a back, a hip, ankles, elbows and hands (FIGS. 8-10).

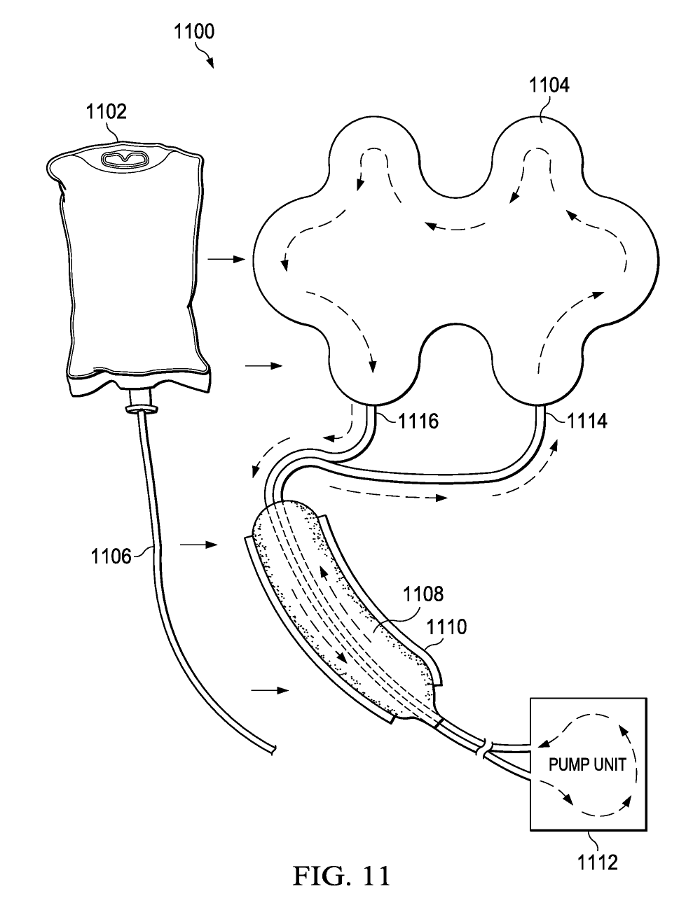

The pad flow design of the present invention may be used to thermally control intravenous (IV) bags, medications, hoses or tubes and designed in the form of a thermal sleeve system. The technology may be used to thermally wrap narrowly or oddly shaped prominences or sharply rounded objects to control temperature. The wrap may be double layered to contain an inner fluid or medication inside but separate from the flow channels but still in contact with the contents and user. (FIG. 11)

The pad of the present invention is designed to fit the curvature of any object internally that may require temperature control. The design of the pad could come in the shape of an artery stint to force open an artery and thermally control the temperature of the artery flow simultaneously. The same could be used inside a bone, organ, brain surface, spinal cord canal for cooling cerebral spinal fluid as seen in FIG. 11.

FIG. 1 shows the multi-dimensional flow pad system 100 of the present invention. The inner surface 102 of the flow pad is in direct contact with the object or the organ and comprises multiple mathematically configured channels 108 and an open face hole 110 for more perfect fitting over pointed areas of the body. The pad 100 is symmetrical about a central axis. There are two hose connectors 104 and 106 attached to the top portion of the pad 100, which may be connected to a pump or a fluid source to provide the heating or cooling fluid that circulates through the channels. The pad 100 is provided with tabs (114, 118, 122, and 126) at specific points along the periphery, these tabs attach to symmetrically placed tabs or points (116, 120, 124 and 128) along the periphery by either a Velcro® arrangement or by hooks or buttons when the pad 100 is curled or placed around the object or organ in question.

FIG. 7 shows the flow pad 700 of the present invention that has multiple flow channels 708 on only one half of the inner surface 702. Hose connector 704 is connected to a fluid source or pump to provide the heating or cooling fluid that flows through the multiple channels 708. Pad 700 comprises an open face hole 710 for more perfect fitting over pointed areas of the body. The pad 700 is provided with tabs (714, 720, 722, and 726) at specific points along the periphery, these tabs attach to symmetrically placed tabs or points (716, 718, 724 and 728) along the periphery by either a Velcro arrangement or by hooks or buttons when the pad 700 is curled or placed around the object or organ in question.

FIG. 8 shows the open face holes 804 in the flow-pad system 800 of the present invention for a more perfect fitting over pointed areas of the body. Flow-pad 800 has an outer surface 802 and hose connectors 806, 808, and 810 for connection to a fluid source or pump. The pad 800 is provided with tabs (814, 816, 820, and 826) at specific points along the periphery, these tabs attach to symmetrically placed tabs or points (812, 818, 822 and 824) along the periphery by either a Velcro arrangement or by hooks or buttons when the pad 800 is curled or placed around the object or organ in question.

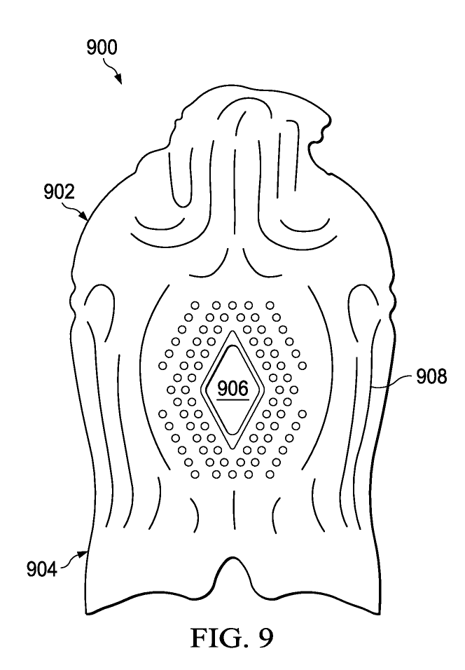

FIG. 9 shows the outer surface of a pad 900 of the present invention covering or wrapped around a three-dimensional body area comprising an upper portion 902, a lower portion 904, and an open face hole 906. Multiple flow channels 908 are present on the inner surface (not shown) of pad 900.

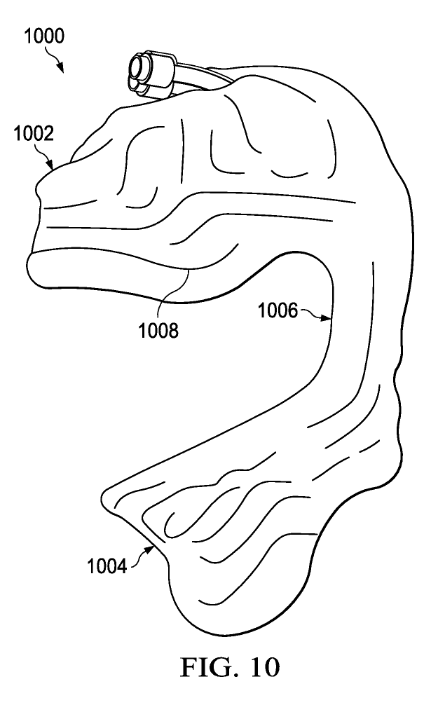

FIG. 10 shows the outer surface of a pad or the wrap 1000 of the present invention showing the upper section 1002, the lower side 1004 and one of the sides 1006. Multiple flow channels 1008 are present on the inner surface (not shown) of pad 1000.

FIG. 11 is a schematic of a set-up 1100 showing the use of the pad 1104 of the present invention to thermally control intravenous (IV) bags or transplant organs 1102 by forming a thermal sleeve 1108. The IV bag or the organ 1102 is placed in the pad 1104, which has an inlet 114 and outlet 1116 that allows for the circulation of the cooling or the heating fluid. The pad 1104 is curled up to enclose the IV bag or the organ 1102 to form the thermal sleeve 1108 and is held together by fasteners 1110. The IV tubing 116 is also contained in the sleeve 1108. The pump or the heating/cooling fluid source is shown by 1112.

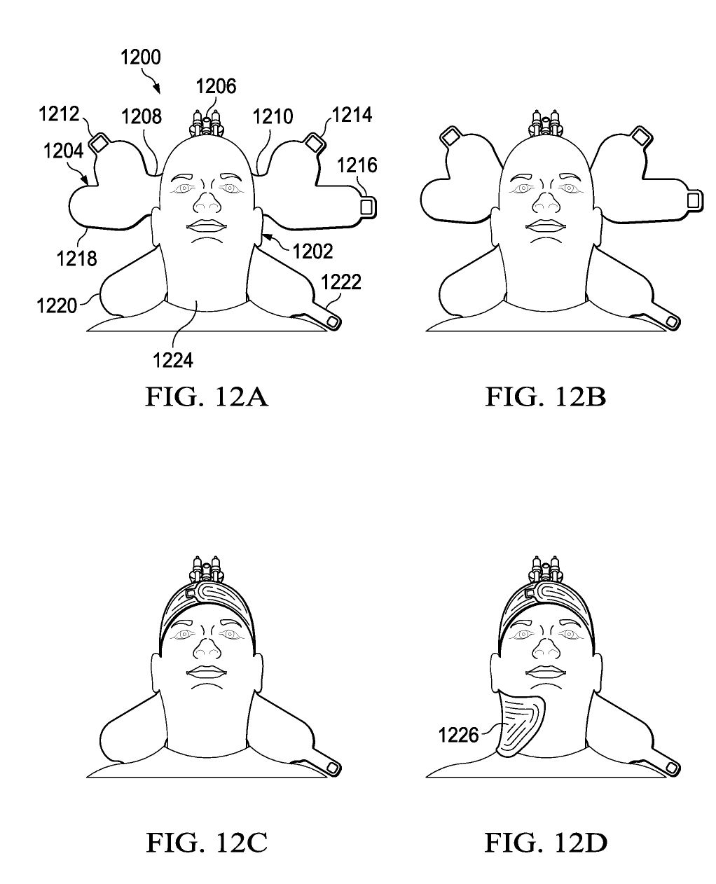

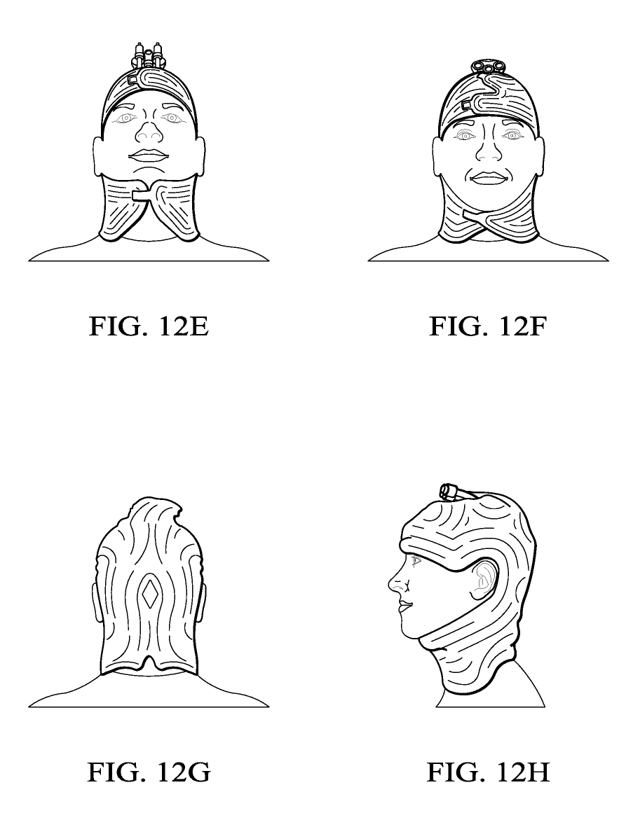

FIGS. 12A-12H show step by step instructions 1200 on the use of the thermal pad system of the present invention to cover the head of the subject. In FIG. 12A the pad 1204 is placed flat behind the head of the subject keeping the hoses 1206 on top of the subject's head (1202). The pad 1204 is adjusted so that the subject's head 1202 is at the center. Tab 1208 is pulled and attached to 1210 (FIG. 12B). Tabs 1212 and 1216 are attached to tabs 1214 and 1218, respectively (FIG. 12C). The “cap” that is formed by the attachment of the tabs is adjusted so that a snug fit was obtained without any creases or impingements. 1220 was gently pulled across the neck 1224 and attached to tab 1222 taking care to see that there are no restrictions or pressure on any airways or blood vessels (FIGS. 12D and 12E). Fitting pad 1204 snugly to form a cap should expose the outer surface 1226 of the pad 1204. The pad 1204 is adjusted with all the

Velcro tabs to obtain a snug but not too tight a fit. The hose connectors 1206 at the top of the pad 1204 are then connected to the connectors from the pump. A front view if a proper fit of the pad 1204 is shown in FIG. 12F. FIGS. 12G and 12H show the corresponding back and side-views, respectively.

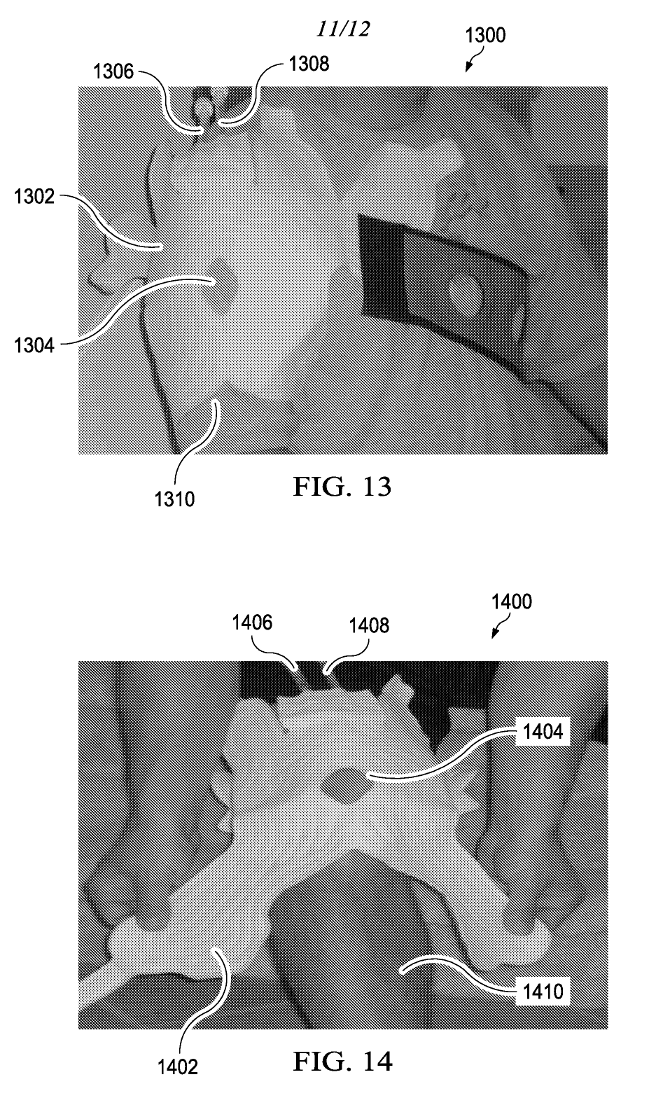

FIG. 13 is a photograph showing the use of the thermal pad system 1300 of the present invention to wrap a shoulder area 1310 of the subject. The outer surface of the pad 1300 is shown by 1302, and the open face hole is shown by 1304. Hose connectors 1306 and 1308 are attached to the top of the pad 1300.

FIG. 14 is a photograph showing the use of the thermal pad system 1400 of the present invention to wrap a knee joint (knee brace) 1410 of the subject. The outer surface of the pad 1400 is shown by 1402, and the open face hole to accommodate the knee is shown by 1404. Hose connectors 1406 and 1408 are attached to the top of the pad 1400.

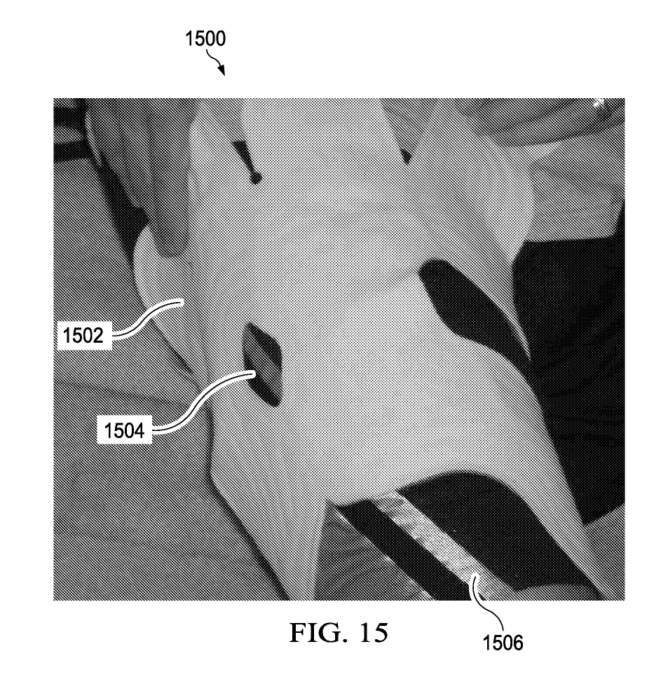

FIG. 15 is a photograph showing the use of the thermal pad system 1500 of the present invention to wrap around the thigh 1506 of the subject. The outer surface of the pad 1500 is shown by 1502, and the open face hole is shown by 1504.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 5,840,397: Sports Pad.

U.S. Pat. No. 5,891,187: Temperature Control Pad for use During Medical and Surgical Procedures.

U.S. Pat. No. 5,894,615: Temperature Selectively Controllable Body Supporting Pad.

U.S. Patent Application No. 20090287060: Methods and Devices to Decrease Tissue Trauma during Surgery.

What is claimed is:

1. A multi-dimensional pad for covering an object, an organ, a tissue, an anatomical part, for insertion into a body organ or tissue or both for providing a temperature control, a temperature therapy, prevent trauma or any combinations thereof comprising:
one or more axisymmetric or similarly shaped surfaces, lobes or panels that are permanently or removeably bonded together to form the pad; wherein the surfaces, lobes or the panels comprise an interior and an exterior, wherein the interior is in direct contact with the object, the organ, the tissue or the anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad to cover the object, the organ, the tissue or the anatomical part.

2. The multi-dimensional pad of claim 1, further comprising:
one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof;
an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part; and
one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part.

3. The multi-dimensional pad of claim 2, wherein the air pad is capable of being inflated to provide pressure to the multi-dimensional pad.

4. The multi-dimensional pad of claim 2, wherein the air pad comprises one or more valves for inflation, deflation or both to provide intermittent or varying pressures to the multi-dimensional pad.

5. The multi-dimensional pad of claim 1, wherein the pad comprises split networks or channels to provide temperature control or localized heating or cooling to specific areas of the organ, the tissue or the anatomical part.

6. The multi-dimensional pad of claim 1, wherein the network is further defined as comprising two networks, wherein the heating fluid and the cooling fluid independently circulate in the two networks concurrently to provide simultaneous heating and cooling to a same or a different area of the organ, the tissue or the anatomical part.

7. The multi-dimensional pad of claim 1, wherein the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof.

8. The multi-dimensional pad of claim 1, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

9. The multi-dimensional pad of claim 1, wherein the anatomical object is selected from the group consisting of an arm, a shoulder, a knee, a head, a neck, a back, a hip, ankles, an elbows and hands.

10. The multi-dimensional pad of claim 1, wherein the multi-dimensional pad provides temperature control to one or more objects selected from the group consisting of intravenous (IV) bags, medications, surgical equipment, hoses, tubes, and any combinations thereof by covering the object to form a thermal sleeve.

11. The multi-dimensional pad of claim 1, wherein the multi-dimensional pad can be inserted into a bone, the organ, a brain surface, and a spinal cord canal for cooling cerebral spinal fluid or for providing temperature control.

12. A multi-dimensional pad for covering an object, an organ, a tissue, an anatomical part, for insertion into a body organ or tissue or both for providing a temperature control, a temperature therapy, prevent trauma or any combinations thereof comprising:
one or more axisymmetric or similarly shaped surfaces, lobes or panels that are permanently or removeably bonded together to form the pad; wherein the surfaces, lobes or the panels comprise an interior and an exterior, wherein the interior is in direct contact with the object, the organ, the tissue or the anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more secondary flow paths, crossovers or bypasses for uninterrupted fluid flow in case of a blockage, pressure build up or obstructions in the primary channels, conduits or tubes;
one or more hose connectors attached to the interior in communication with the network, wherein the hose connectors are connected to and deliver the heating or the cooling fluid from a pump or a fluid reservoir to the network;
one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof;
an air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part;
one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part; and
one or more fasteners, wherein the fasteners are capable of fastening on to other fasteners or regions on the interior or exterior of the pad to cover the object, the organ, the tissue or the anatomical part.

13. A method for providing a controlled temperature environment to an organ, a tissue or an anatomical part of a subject comprising the steps of:
identifying the subject in need of the controlled temperature environment;
providing a multi-dimensional pad comprising:
one or more axisymmetric or similarly shaped surfaces, lobes or panels that are permanently or removeably bonded together to form the pad, wherein the surfaces, lobes or the panels comprise an interior and an exterior, wherein the interior is in direct contact with the object, the organ, the tissue or the anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad to cover the object, the organ, the tissue or the anatomical part;
placing the organ, the tissue or the anatomical part in the multidimensional pad or covering, wrapping or overlaying the multidimensional pad over the organ, the tissue or the anatomical part such that the interior is in direct contact with the organ, the tissue or the anatomical part;
fastening the one or more fasteners on the interior of the pad to one or more fasteners or regions on the interior or exterior of the pad to obtain a snug fit of the multidimensional pad over the organ, the tissue or the anatomical part; and
providing a controlled temperature by operating the pump or the fluid reservoir to deliver the circulating heating or cooling fluid to the network through the one or more hose connectors and associated tubing.

14. The method of claim 13, wherein the multi-dimensional further comprises:
one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof;
an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the organ, the tissue or the anatomical part; and
one or more additional layers attached removeably or permanently to the multi-dimensional pad comprising air, fluid, medication or any combinations thereof, wherein the additional layers are separate from the flow channels and are in direct contact with the organ, the tissue or the anatomical part.

15. The method of claim 14, wherein the air pad is capable of being inflated to provide pressure to the multi-dimensional pad.

16. The method of claim 14, wherein the air pad comprises one or more valves for inflation, deflation or both to provide intermittent or varying pressures to the multi-dimensional pad.

17. The method of claim 13, wherein the multi-dimensional pad curls up to provide a more snug fit over the object, the organ, the tissue or the anatomical part when fluid circulation is initiated.

18. The method of claim 13, wherein the pad comprises split networks or channels to provide temperature control or localized heating or cooling to specific areas of the organ, the tissue or the anatomical part.

19. The method of claim 13, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

20. The method of claim 13, wherein the network is further defined as comprising two networks, wherein the heating fluid and the cooling fluid independently circulate in the two networks concurrently to provide simultaneous heating and cooling to a same or a different area of the organ, the tissue or the anatomical part.

21. The method of claim 13, wherein the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof.

22. The method of claim 13, wherein the anatomical object is selected from the group consisting of an arm, a shoulder, a knee, a head, a neck, a back, a hip, ankles, an elbows and hands.

23. The method of claim 13, wherein the multi-dimensional pad can be inserted into a bone, the organ, a brain surface, and a spinal cord canal for cooling cerebral spinal fluid or for providing temperature control.

24. A method for providing a controlled temperature environment to an object comprising the steps of:
providing a thermal sleeve comprising:
one or more axisymmetric or similarly shaped surfaces, lobes or panels that are permanently or removeably bonded together to form the sleeve, wherein the surfaces, lobes or the panels comprise an interior and an exterior, wherein the interior is in direct contact with the object, the organ, the tissue or the anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are selected from the group consisting of Velcro fasteners, hooks and loops, button, pin, and any combinations thereof;
placing the object in the thermal sleeve or covering, wrapping or overlaying the thermal sleeve over the such that the interior is in direct contact with the object;
fastening the one or more fasteners on the interior of the sleeve to one or more fasteners or regions on the interior or exterior of the pad to obtain a snug fit of the sleeve over the object; and
providing a controlled temperature by operating the pump or the fluid reservoir to deliver the circulating heating or cooling fluid to the network through the one or more hose connectors and associated tubing.

25. The method of claim 24, wherein the thermal sleeve further comprises:
one or more open-face holes, curves, lobe like designs or any combinations thereof on the surface, lobes or the panels, wherein the open-face holes, curves, lobe like designs assist in a fitting of the sleeve on oddly shaped objects, projections, protrusions or any combinations thereof;
an optional air-flow mechanism comprising one or more secondary flow channels or an air pad attached removeably or permanently to the thermal sleeve, wherein the secondary flow channel or the air-pad provides a sustained, intermittent or varying pressures to the thermal sleeve to enhance contact of the multi-dimensional pad with the object; and
one or more additional layers attached removeably or permanently to the thermal sleeve comprising air, fluid, medication or any combinations thereof, wherein the additional layers are separate from the flow channels and are in direct contact with the object.

26. The method of claim 25, wherein the air pad is capable of being inflated to provide pressure to the thermal sleeve.

27. The method of claim 25, wherein the air pad comprises one or more valves for inflation, deflation or both to provide intermittent or varying pressures to the thermal sleeve.

28. The method of claim 24, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

29. The method of claim 24, wherein the object is selected from the group consisting of intravenous (IV) bags, medications, surgical equipment, hoses, tubes, and any combinations thereof

30. A method of preventing or minimizing brain trauma following a head injury, a brain surgery, a stroke, an aneurysm, a hemorrhage or any combinations thereof in a subject comprising the steps of:
identifying the subject in need of prevention or minimization of the brain trauma;
placing the head of the subject or placing a multi-dimensional pad under the head of the subject, wherein the position of the pad is such that the head of the subject is at a center of the pad, wherein the pad comprises:
one or more axisymmetric or similarly shaped surfaces, lobes or panels that are permanently or removeably bonded together to form the pad, wherein the surfaces, lobes or the panels comprise an interior and an exterior, wherein the interior is in direct contact with the head;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad to cover the head;
fastening the one or more fasteners on the interior of the pad to one or more fasteners or regions on the interior or exterior of the pad to obtain a snugly fit cap of the multidimensional pad covering the head and neck of the subject; and
operating the pump or the fluid reservoir to deliver the circulating heating or cooling fluid to the network through the one or more hose connectors and associated tubing to provide a controlled temperature environment to minimize or prevent the brain trauma.

31. The method of claim 30, wherein the pad further comprises:
one or more open-face holes, curves, lobe like designs or any combinations thereof on the surface, lobes or the panels, wherein the open-face holes, curves, lobe like designs assist in a fitting of the pad to the head;
an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the head; and
one or more additional layers attached removeably or permanently to the multi-dimensional pad comprising air, fluid, medication or any combinations thereof, wherein the additional layers are separate from the flow channels and are in direct contact with the head.

32. The method of claim 31, wherein the air pad is capable of being inflated to provide pressure to the thermal sleeve.

33. The method of claim 31, wherein the air pad comprises one or more valves for inflation, deflation or both to provide intermittent or varying pressures to the thermal sleeve.

34. The method of claim 30, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

35. A method of minimizing or preventing degradation of an organ during harvesting, transporting, transplanting into a patient or any combinations thereof comprising:
placing the organ in a multi-dimensional pad or thermal sleeve, wherein the pad or the sleeve comprises:
one or more axisymmetric or similarly shaped surfaces, lobes or panels that are permanently or removeably bonded together to form the pad or the sleeve, wherein the surfaces, lobes or the panels comprise an interior and an exterior, wherein the interior is in direct contact with the object, the organ, the tissue or the anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad or the sleeve to cover the organ;
fastening the one or more fasteners to obtain a snug fit around the organ to cover or overlay the organ; and
operating the pump or the fluid reservoir to deliver the circulating cooling fluid to the network through the one or more hose connectors to provide a controlled temperature environment to minimize or prevent degradation during organ harvest, transport, transplant or any combinations thereof.

36. The method of claim 35, the multi-dimensional pad or the thermal sleeve further comprises:
one or more open-face holes, curves, lobe like designs or any combinations thereof on the surface, lobes or the panels, wherein the open-face holes, curves, lobe like designs assist in a fitting of the pad or the sleeve on oddly shaped organs, projections, protrusions or any combinations thereof;
an optional air-flow mechanism comprising one or more secondary flow channels or an air pad attached removeably or permanently to the thermal sleeve, wherein the secondary flow channel or the air-pad provides a sustained, intermittent or varying pressures to the thermal sleeve to enhance contact of the pad or the sleeve with the organ; and
one or more additional layers attached removeably or permanently to the pad or the thermal sleeve comprising air, fluid, medication or any combinations thereof, wherein the additional layers are separate from the flow channels and are in direct contact with the organ.

37. A system for providing a controlled temperature environment during a surgical procedure, in an operation theater, in an emergency room or any combinations thereof comprising one or more thermal sleeves and multi-dimensional pads, wherein the thermal sleeves and multi-dimensional pads comprise:
one or more axisymmetric or similarly shaped surfaces, lobes or panels that are permanently or removeably bonded together to form the pad, wherein the surfaces, lobes or the panels comprise an interior and an exterior, wherein the interior is in direct contact with an object, an organ, a tissue or an anatomical part;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad to cover or overlay the object, the organ, the tissue or the anatomical part.

38. The system of claim 37, further comprising:
one or more open-face holes, curves, lobe like designs or any combinations thereof on the surface, lobes or the panels, wherein the open-face holes, curves, lobe like designs assist in a fitting of the pad on oddly shaped objects, projections, protrusions or any combinations thereof;
an optional air-flow mechanism comprising one or more secondary flow channels or an air pad attached removeably or permanently to the multi-dimensional pad, wherein the secondary flow channel or the air-pad provides a sustained, intermittent or varying pressures to the multi-dimensional pad to enhance contact of the multi-dimensional pad with the object, the organ, the tissue or the anatomical part; and
one or more additional layers attached removeably or permanently to the multi-dimensional pad comprising air, fluid, medication or any combinations thereof, wherein the additional layers are separate from the flow channels and are in direct contact with the object, the organ, the tissue or the anatomical part.

39. The system of claim 38, wherein the air pad is capable of being inflated to provide pressure to the multi-dimensional pad.

40. The system of claim 38, wherein the air pad comprises one or more valves for inflation, deflation or both to provide intermittent or varying pressures to the multi-dimensional pad.

41. The system of claim 37, wherein the pad comprises split networks or channels to provide temperature control or localized heating or cooling to specific areas of the organ, the tissue or the anatomical part.

42. The system of claim 37, wherein the one or more channels, conduits or tubes are designed to make sharp turns and comprise one or more secondary flow paths, crossovers or bypasses for uninterrupted fluid flow in case of a blockage, pressure build up or obstructions in the primary channels, conduits or tubes.

43. The system of claim 37, wherein the multi-dimensional pad covers or overlays one or more anatomical objects selected from the group consisting of an arm, a shoulder, a knee, a head, a neck, a back, a hip, ankles, an elbows and hands during a surgical procedure.

44. The system of claim 37, wherein the thermal sleeve covers or overlays one or more objects selected from the group consisting of intravenous (IV) bags, medications, surgical equipment, hoses, tubes, and any combinations thereof.

45. The system of claim 37, wherein the multi-dimensional pad can be inserted into a bone, the organ, a brain surface, and a spinal cord canal for cooling cerebral spinal fluid or for providing temperature control during a surgical procedure.

46. A method of providing a controlled temperature for one or more surgical and associated equipments, organs, tissues, and any combinations thereof during a surgical procedure comprising the step of wrapping, covering or overlaying with a multi-dimensional pad or a thermal sleeve.

47. The method of claim 46, wherein the multi-dimensional pad or thermal sleeve comprises:
one or more axisymmetric or similarly shaped surfaces, lobes or panels that are permanently or removeably bonded together to form the pad, wherein the surfaces, lobes or the panels comprise an interior and an exterior, wherein the interior is in direct contact with the one or more surgical and associated equipments organs, tissues, and any combinations thereof;
one or more primary channels, conduits, tubes or any combinations thereof removeably or permanently attached or embedded to the interior in fluid communication to form a network, wherein the channels, the conduits or the tubes comprise a circulating heating or a cooling fluid and are capable of a change in a height and a diameter based on a volume of the circulating heating or a cooling fluid;
one or more hose connectors attached to the interior in communication with the network, wherein the hose connectors are connected to and deliver the heating or the cooling fluid from a pump or a fluid reservoir to the network; and
one or more fasteners, wherein the fasteners are capable of fastening on to other fasteners or regions on the interior or exterior of the pad to cover the one or more surgical and associated equipments, organs, tissues, and any combinations thereof.

48. The method of claim 46, wherein the one or more surgical and associated equipments are selected from the group consisting of intravenous (IV) bags, medications, surgical equipment, hoses, tubes, and any combinations thereof.

49. The method of claim 46, wherein the multi-dimensional pad covers or overlays one or more anatomical objects selected from the group consisting of an arm, a shoulder, a knee, a head, a neck, a back, a hip, ankles, an elbows and hands or can be inserted into a bone, the organ, a brain surface, and a spinal cord canal for cooling cerebral spinal fluid or for providing temperature control during a surgical procedure.

50. The method of claim 46, wherein the one or more fasteners selected from the group consisting of Velcro fasteners, hooks and loops, button, pin, and any combinations thereof, wherein the fasteners are removeably attached at any location of the pad, are integral to the multi-dimensional pad, are attached to tabs extending from a periphery of the multi-dimensional pad or any combinations thereof.
